(19) United States
(12) Patent Application Publication
(10) Pub. No.: US 2024/0335281 A1
(43) Pub. Date: Oct. 10, 2024

(54) REPLACEMENT HEART VALVE IMPLANT AND A TOOL AND METHOD FOR ASSEMBLING A REPLACEMENT HEART VALVE IMPLANT

(71) Applicant: BOSTON SCIENTIFIC SCIMED, INC., Maple Grove, MN (US)

(72) Inventors: Tim O'Connor, Galway (IE); Pearse A Coffey, Galway (IE); Declan Loughnane, Galway (IE); Joseph Murphy, Galway (IE)

(73) Assignee: BOSTON SCIENTIFIC SCIMED, INC., MAPLE GROVE, MN (US)

(21) Appl. No.: 18/625,602

(22) Filed: Apr. 3, 2024

Related U.S. Application Data
(60) Provisional application No. 63/456,853, filed on Apr. 4, 2023.

(51) Int. Cl. A61F 2/24

(52) U.S. Cl. CPC A61F 2/2418; A61F 2/2415; A61F 2210/0014; A61F 2220/0025

(57) ABSTRACT

A replacement heart valve implant includes an expandable framework including a commissure post and defining a central lumen, and a valve leaflet disposed within the central lumen, the valve leaflet including a mounting tab secured to the expandable framework at the commissure post. The commissure post is configured to hold the mounting tab in compression. A tool for assembling a replacement heart valve implant includes a tool body comprising first and second body portions, first and second handle portions extending from the body portions, and first and second post engagement arms extending from the body portions. Translation of the first handle portion toward the second handle portion translates the first post engagement arm away from the second post engagement arm.

### CROSS-REFERENCE TO RELATED APPLICATIONS

This application claims the benefit of priority of U.S. Provisional Application No. 63/456,853 filed Apr. 4, 2023, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve implant, a tool for assembling a replacement heart valve implant, and a method of assembling a replacement heart valve implant.

### BACKGROUND

A wide variety of intracorporeal medical devices have been developed for medical use, for example, surgical and/or intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, a replacement heart valve implant may comprise an expandable framework including a commissure post and defining a central lumen extending axially through the expandable framework, and a valve leaflet disposed within the central lumen of the expandable framework. The valve leaflet may include a mounting tab extending therefrom. The mounting tab may be secured to the expandable framework at the commissure post. The commissure post may be configured to hold the mounting tab in compression.

In addition or alternatively to any example described herein, the expandable framework is formed from a resilient shape memory material.

In addition or alternatively to any example described herein, the commissure post includes an elongated aperture at least partially defined by a first post side and a second post side, the elongated aperture having an axial extent and a circumferential extent, the axial extent being greater than the circumferential extent.

In addition or alternatively to any example described herein, the mounting tab extends through the elongated aperture.

In addition or alternatively to any example described herein, the circumferential extent is configured to be shifted between a first circumferential dimension between the first post side and the second post side, and a second circumferential dimension between the first post side and the second post side, wherein the second circumferential dimension is greater than the first circumferential dimension.

In addition or alternatively to any example described herein, the commissure post is self-biased toward the first circumferential dimension.

In addition or alternatively to any example described herein, the second circumferential dimension is greater than a thickness of the mounting tab.

In addition or alternatively to any example described herein, the replacement heart valve implant may further comprise a second valve leaflet disposed within the central lumen of the expandable framework. The second valve leaflet may include a second mounting tab extending therefrom.

In addition or alternatively to any example described herein, the second mounting tab is secured to the expandable framework at the commissure post.

In addition or alternatively to any example described herein, the commissure post is configured to hold the second mounting tab in compression.

In addition or alternatively to any example described herein, the mounting tab and the second mounting tab are in intimate contact with each other at the commissure post.

In addition or alternatively to any example described herein, a tool for assembling a replacement heart valve implant may comprise a tool body comprising a first body portion and a second body portion rotatably engaged with the first body portion, a first handle portion extending from the first body portion, the first handle portion being configured to rotate about an axis of rotation, a second handle portion extending from the second body portion, the second handle portion being configured to rotate about the axis of rotation, a first post engagement arm extending from the first body portion, the first post engagement arm being configured to rotate about the axis of rotation, and a second post engagement arm extending from the second body portion, the second post engagement arm being configured to rotate about the axis of rotation. Translation of the first handle portion toward the second handle portion may translate the first post engagement arm away from the second post engagement arm.

In addition or alternatively to any example described herein, the first handle portion is biased away from the second handle portion.

In addition or alternatively to any example described herein, the tool is configured to shift between a first configuration wherein the first post engagement arm and the second post engagement arm are configured to engage a commissure post of an expandable framework of the replacement heart valve implant, and a second configuration wherein the first post engagement arm and the second post engagement arm are configured to expand an elongate aperture formed in the commissure post in a circumferential direction.

In addition or alternatively to any example described herein, the tool is self-biased toward the first configuration.

In addition or alternatively to any example described herein, the tool may further comprise a spring disposed within the tool body.

In addition or alternatively to any example described herein, the spring biases the tool toward the first configuration.

In addition or alternatively to any example described herein, the tool may further comprise a ratchet mechanism configured to lock the tool in the second configuration.

In addition or alternatively to any example described herein, the tool may further comprise a release knob rotatably engaged with the tool body.

In addition or alternatively to any example described herein, the release knob may be configured to disengage the ratchet mechanism, thereby unlocking the tool from the second configuration.

In addition or alternatively to any example described herein, a method of assembling a replacement heart valve implant may comprise: obtaining an expandable framework including a commissure post and defining a central lumen extending axially through the expandable framework; obtaining a valve leaflet including a mounting tab extending therefrom; translating a first post side of the commissure post circumferentially away from a second post side of the commissure post to expand an elongated aperture formed in the commissure post from a first circumferential dimension to a second circumferential dimension; positioning the valve leaflet within the central lumen; extending the mounting tab from the central lumen radially outward through the elongated aperture while the elongated aperture is held at the second circumferential dimension; and translating the first post side of the commissure post circumferentially toward the second post side of the commissure post to compress the mounting tab between the first post side and the second post side.

In addition or alternatively to any example described herein, after translating the first post side of the commissure post circumferentially toward the second post side of the commissure post, the mounting tab is secured to the commissure post only by compression. In addition or alternatively to any example described herein, the method may further comprise trimming a portion of the mounting tab extending radially outward of the commissure post.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects an expandable framework associated with the replacement heart valve implant of FIG. 1;

FIGS. 3-5 illustrate selected aspects of a tool for assembling the replacement heart valve implant of FIG. 1; and

FIGS. 6-14 illustrate selected aspects related to using the tool of FIGS. 3-5 to assemble the replacement heart valve implant of FIG. 1.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all of the components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

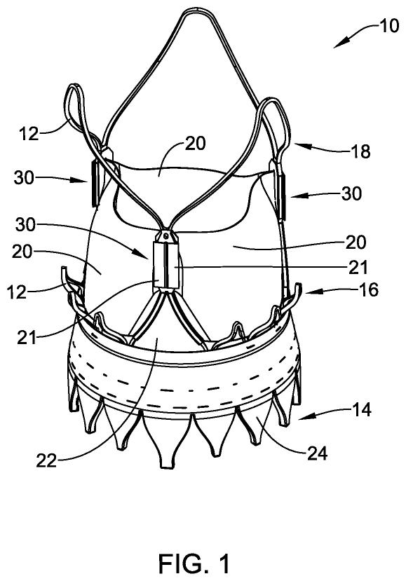

FIG. 1 illustrates selected aspects of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 may be configured to shift between a radially collapsed configuration and a radially expanded configuration. In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the expandable framework 12 may include a tubular wall defining the central lumen, the inflow end, the outflow end, the lower crown 14, and/or the upper crown 16. In some embodiments, the plurality of stabilization arches 18 may extend downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the expandable framework 12 may include a commissure post 30. In some embodiments, the expandable framework 12 may include a plurality of commissure posts. In at least some embodiments, the commissure post 30 and/or the plurality of commissure posts may be disposed longitudinally and/or axially between the upper crown 16 and the plurality of stabilization arches 18. In some embodiments, the plurality of stabilization arches 18 may extend from the commissure post 30 and/or the plurality of commissure posts.

In some embodiments, the replacement heart valve implant 10 may include a proximal portion and a distal portion. In some embodiments, orientation of the replacement heart valve implant 10 may be related to an implant delivery device and/or a direction of implantation relative to a target site. In some embodiments, the proximal portion may include the outflow end and/or the plurality of stabilization arches 18. In some embodiments, the proximal portion may include the upper crown 16 and/or the commissure post 30 and/or the plurality of commissure posts. In some embodiments, the distal portion may include the inflow end and/or the lower crown 14. Other configurations are also contemplated.

In some embodiments, the replacement heart valve implant 10 may include a valve leaflet 20 disposed within the central lumen of the expandable framework 12. In some embodiments, the replacement heart valve implant 10 may include a second valve leaflet disposed within the central lumen of the expandable framework 12. In some embodiments, the replacement heart valve implant 10 may include a plurality of valve leaflets disposed within the central lumen. The valve leaflet 20, the second valve leaflet, and/or the plurality of valve leaflets may be coupled, secured, and/or fixedly attached to the expandable framework 12. In some embodiments, the outflow end of the expandable framework 12 may include the plurality of stabilization arches 18 extending axially away from the valve leaflet 20, the second valve leaflet, and/or the plurality of valve leaflets and/or from the commissure post 30 (or the plurality of commissure posts) or an attachment point (or attachment points) of the valve leaflet 20, the second valve leaflet, and/or the plurality of valve leaflets with the expandable framework 12.

The valve leaflet 20 and/or the plurality of valve leaflets may include a mounting tab 21 extending therefrom. The second valve leaflet may include a second mounting tab extending therefrom. In some embodiments, the valve leaflet 20, the second valve leaflet, and/or the plurality of valve leaflets may include a mounting tab 21 extending from each end thereof. For example, in at least some embodiments, the valve leaflet 20, the second valve leaflet, and/or the plurality of valve leaflets may include two mounting tabs at opposite ends thereof. The mounting tab 21, the second mounting tab, and/or the mounting tabs may be secured to the expandable framework 12 at the commissure post 30 and/or the plurality of commissure posts.

In some embodiments, each valve leaflet of the plurality of valve leaflets may include a root edge coupled to the expandable framework 12 and a free edge (e.g., a coaptation edge) movable relative to the root edge to coapt with the free edges of the other leaflets along a coaptation region. In some embodiments, the plurality of valve leaflets can be integrally formed with each other, such that the plurality of valve leaflets is formed as a single unitary and/or monolithic unit. In some embodiments, the valve leaflet 20, the second valve leaflet, and/or the plurality of valve leaflets may be formed integrally with other structures such as an inner skirt 22 and/or an outer skirt 24, base structures, liners, or the like.

The valve leaflet 20, the second valve leaflet, and/or the plurality of valve leaflets may be configured to substantially restrict fluid from flowing through the replacement heart valve implant 10 in a closed position. For example, in some embodiments, the free edges of the plurality of valve leaflets may move into coaptation with one another in the closed position to substantially restrict fluid from flowing through the replacement heart valve implant 10. The free edges of the plurality of valve leaflets may be moved apart from each other in an open position to permit fluid flow through the replacement heart valve implant 10. In FIG. 1, the valve leaflet 20, the second valve leaflet, and/or the plurality of valve leaflets is shown in the open position or in a partially open position (e.g., a neutral position) that the valve leaflet 20, the second valve leaflet, and/or the plurality of valve leaflets may move to when unbiased by fluid flow.

In some embodiments, the valve leaflet 20, the second valve leaflet, and/or the plurality of valve leaflets may be comprised of a polymer, such as a thermoplastic polymer. In some embodiments, the valve leaflet 20, the second valve leaflet, and/or the plurality of valve leaflets may include at least 50 percent by weight of a polymer. In some embodiments, the valve leaflet 20, the second valve leaflet, and/or the plurality of valve leaflets may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the valve leaflet 20, the second valve leaflet, and/or the plurality of valve leaflets. In at least some embodiments, the inner skirt 22 may be fixedly attached to and/or integrally formed with the valve leaflet 20 and/or the plurality of valve leaflets. The inner skirt 22 may ensure the fluid flows through the central lumen of the replacement heart valve implant 10 and does not flow around the valve leaflet 20, the second valve leaflet, and/or the plurality of valve leaflets when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the valve leaflet 20, the second valve leaflet, and/or the plurality of valve leaflets can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments one or more of the valve leaflet 20, the second valve leaflet, the plurality of valve leaflets, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the replacement heart valve implant 10 may include a scaling member disposed on the expandable framework 12 proximate the inflow end. In some embodiments, the sealing member may include and/or may be the inner skirt 22. In some embodiments, the sealing member may include and/or may be the outer skirt 24. In some embodiments, the sealing member may include and/or may be the inner skirt 22 and the outer skirt 24. Other configurations are also contemplated.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

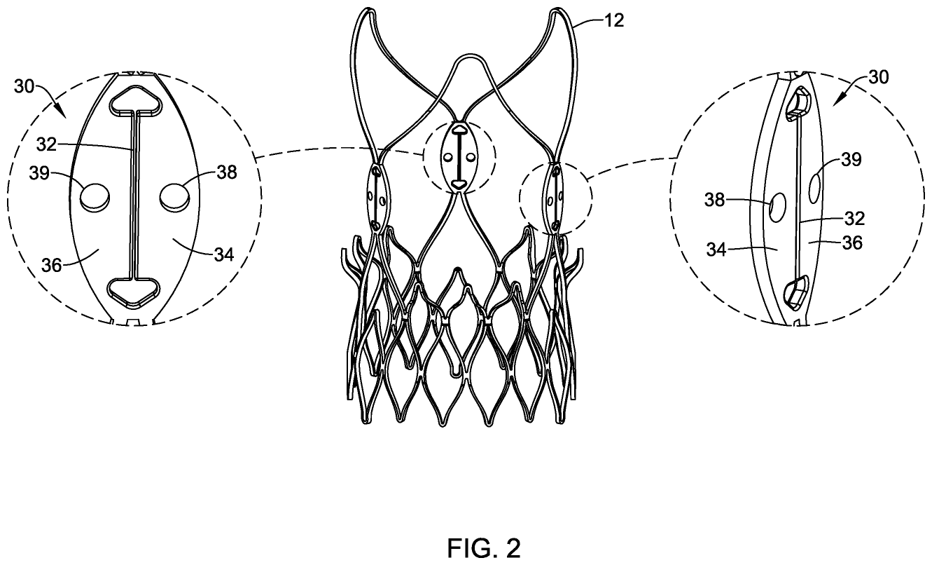

FIG. 2 illustrates selected aspects of the expandable framework 12, with other elements of the replacement heart valve implant 10 removed for clarity. In some embodiments, the expandable framework 12 may include a commissure post 30 and/or a plurality of commissure posts. The detailed views of FIG. 2 show the commissure post 30 and/or the plurality of commissure posts may include an elongated aperture 32 at least partially defined by a first post side 34 and a second post side 36, as viewed from outside the expandable framework 12 looking inward toward the central lumen. As viewed from outside the expandable framework 12 looking inward toward the central lumen, the first post side 34 may be disposed left of the elongated aperture 32 and the second post side 36 may be disposed right of the elongated aperture 32.

In at least some embodiments, the elongated aperture 32 may be oriented axially and/or longitudinally. In some embodiments, the elongated aperture 32 may have an axial extent and a circumferential extent (or a lateral extent). The axial extent may be greater than the circumferential extent or the lateral extent. The commissure post 30 and/or the plurality of commissure posts may include a first aperture 38 formed in and/or extending through the first post side 34 and a second aperture 39 formed in and/or extending through the second post side 36. In at least some embodiments, the commissure post 30 and/or the plurality of commissure posts may include reliefs and/or cutouts formed therein at and/or adjacent to axial and/or opposing ends of the elongated aperture 32. In some embodiments, the reliefs and/or cutouts may provide and/or promote a desired degree of flexibility and/or resiliency within the commissure post 30 and/or the plurality of commissure posts.

In some embodiments, the expandable framework 12 and/or the commissure post and/or the plurality of commissure posts may be formed from a resilient shape memory material. In at least some embodiments, the resilient shape memory material may be a metallic material such as nitinol. Some suitable but non-limiting examples of materials that may be used to form the expandable framework 12 and/or the commissure post 30 and/or the plurality of commissure posts including but not limited to metallics, polymers, composites, and the like, are described below.

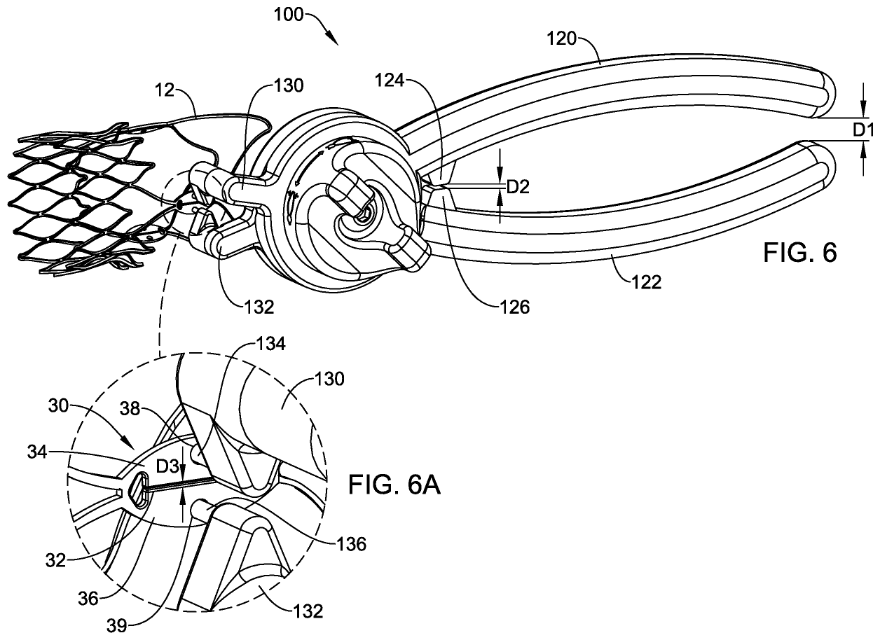

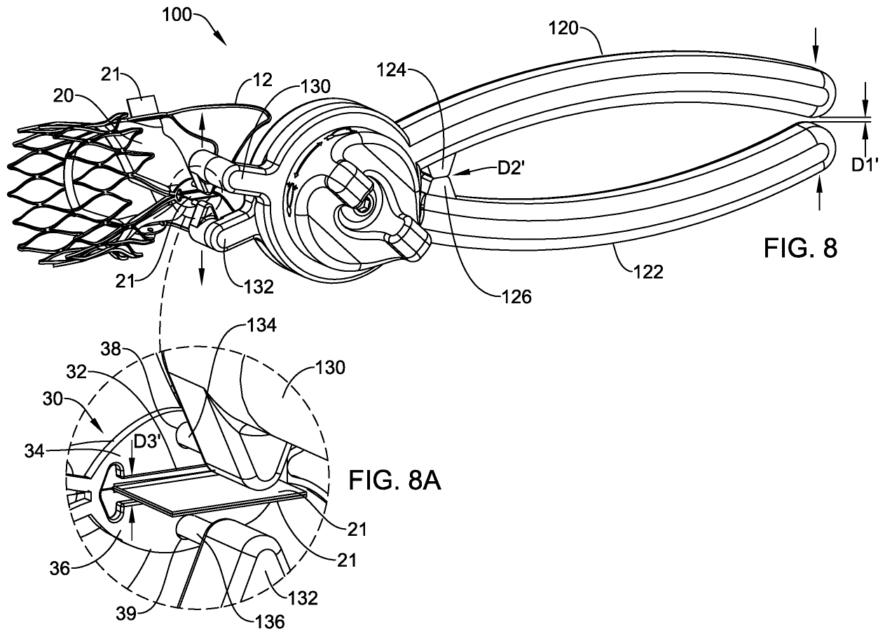

In some embodiments, the circumferential extent of the elongated aperture 32 may be configured to be shifted between a first circumferential dimension D3 (e.g., FIG. 6A) between the first post side 34 and the second post side 36 and a second circumferential dimension D3′ (e.g., FIG. 8A) between the first post side 34 and the second post side 36. The second circumferential dimension may be greater than the first circumferential dimension. In at least some embodiments, the commissure post 30 and/or the plurality of commissure posts may be self-biased toward the first circumferential dimension.

In some embodiments, the second circumferential dimension may be greater than a thickness of the mounting tab 21, the second mounting tab, and/or the mounting tabs. In some embodiments, the second circumferential dimension may be greater than twice the thickness of the mounting tab 21, the second mounting tab, and/or the mounting tabs. In some embodiments, the second circumferential dimension may be greater than a sum of the thickness of the mounting tab 21 and the thickness of the second mounting tab. Other configurations are also contemplated.

In some embodiments, the thickness of the mounting tab 21, the second mounting tab, and/or the mounting tabs may be between about 0.22 millimeters and about 0.27 millimeters. Other configurations are also contemplated. In some embodiments, the first circumferential dimension may be between about 0.30 millimeters and about 0.40 millimeters. In some embodiments, the second circumferential dimension may be between about 0.45 millimeters and about 0.65 millimeters. In some embodiments, the second circumferential dimension may be between about 0.48 millimeters and about 0.60 millimeters. In some embodiments, the second circumferential dimension may be between about 0.50 millimeters and about 0.55 millimeters. Other configurations are also contemplated.

Returning briefly to FIG. 1, in some embodiments, the mounting tab 21 may be secured to the expandable framework 12 at the commissure post 30. In some embodiments, the second mounting tab may be secured to the expandable framework 12 at the commissure post 30. In some embodiments, the mounting tab 21 and the second mounting tab may be secured to the expandable framework 12 at the commissure post 30. In some embodiments, the mounting tabs may be secured to the expandable framework 12 at the plurality of commissure posts. In some embodiments, the mounting tab 21 of the valve leaflet 20 may extend through the elongated aperture 32. In some embodiments, the second mounting tab of the second valve leaflet may extend through the elongated aperture 32. In some embodiments, the mounting tab 21 and the second mounting tab may be in intimate contact with each other at the commissure post 30. In some embodiments, two mounting tabs (one mounting tab from each of two different valve leaflets) may be in intimate contact with each other at each commissure post of the plurality of commissure posts.

In at least some embodiments, edges of the elongated aperture 32 and/or the reliefs or cutouts disposed at and/or adjacent axial and/or opposing ends of the elongated aperture 32 may be rounded to prevent damage to the mounting tab 21, the second mounting tab, and/or the mounting tabs engaged therewith, as seen in the detail views of FIG. 2 for example. In some embodiments, interior facing (e.g., toward the central lumen) edges of the elongated aperture 32 and/or the reliefs or cutouts disposed at and/or adjacent axial and/or opposing ends of the elongated aperture 32 may be rounded to prevent damage to the mounting tab 21, the second mounting tab, and/or the mounting tabs engaged therewith. In some embodiments, exterior facing edges (e.g., away from the central lumen) of the elongated aperture 32 and/or the reliefs or cutouts disposed at and/or adjacent axial and/or opposing ends of the elongated aperture 32 may be rounded to prevent damage to the mounting tab 21, the second mounting tab, and/or the mounting tabs engaged therewith. In some embodiments, both interior facing edges and exterior facing edges of the elongated aperture 32 and/or the reliefs or cutouts disposed at and/or adjacent axial and/or opposing ends of the elongated aperture 32 may be rounded to prevent damage to the mounting tab 21, the second mounting tab, and/or the mounting tabs engaged therewith. In some embodiments, all edges of the elongated aperture 32 and/or the reliefs or cutouts disposed at and/or adjacent axial and/or opposing ends of the elongated aperture 32 may be rounded to prevent damage to the mounting tab 21, the second mounting tab, and/or the mounting tabs engaged therewith.

In some embodiments, the commissure post 30 may be configured to hold the mounting tab 21 of the valve leaflet 20 in compression. For example, when the circumferential extent is at the first circumferential dimension, the first post side 34 and the second post side 36 may squeeze and/or compress the mounting tab 21 of the valve leaflet 20 therebetween. In some embodiments, the commissure post 30 may be configured to hold the second mounting tab of the second valve leaflet in compression. In some embodiments, the commissure post 30 may be configured to hold the mounting tab 21 of the valve leaflet 20 and the second mounting tab of the second valve leaflet in compression. In some embodiments, the commissure post 30 and/or the plurality of commissure posts may each be configured to hold two mounting tabs (one mounting tab from each of two different valve leaflets) in compression.

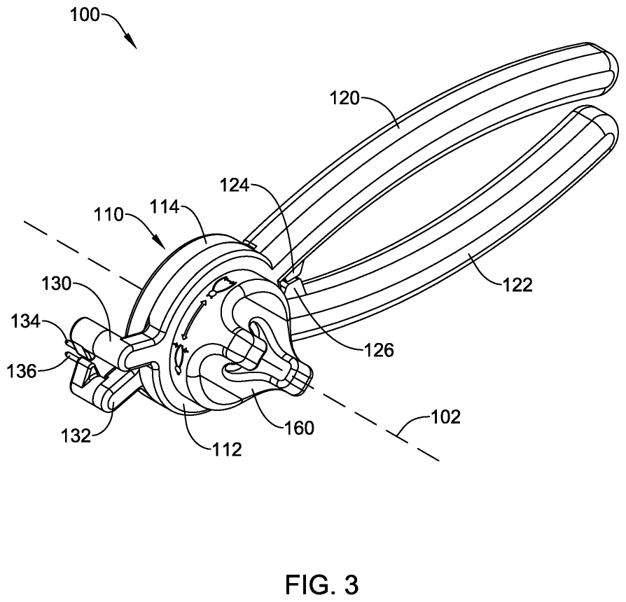

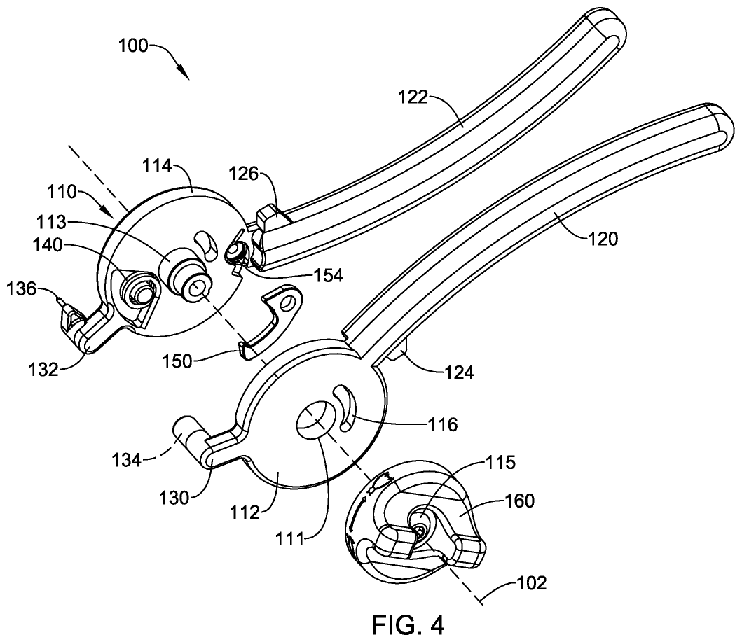

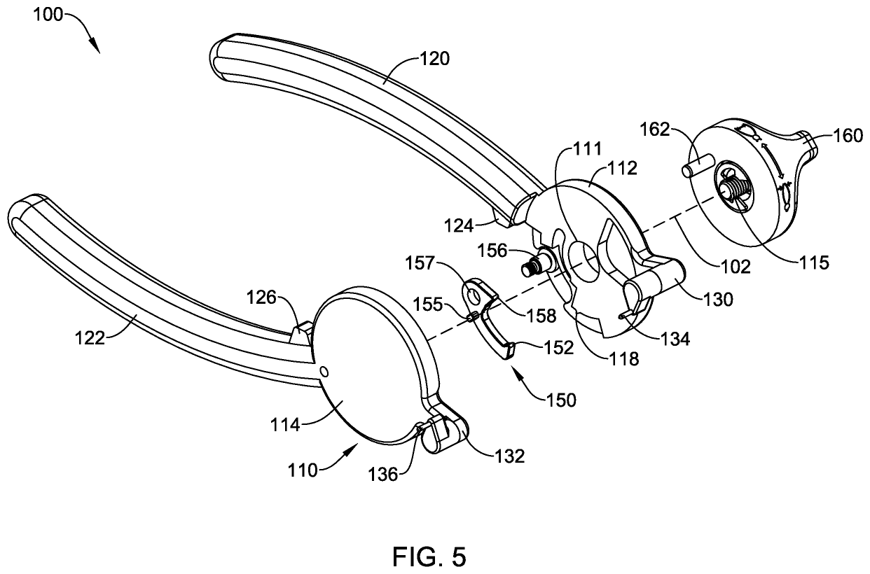

FIGS. 3-5 illustrate selected aspects of a tool 100 for assembling the replacement heart valve implant 10, wherein FIGS. 4-5 are partially exploded views of the tool 100. The tool 100 may comprise a tool body 110 comprising a first body portion 112 and a second body portion 114 movably engaged with the first body portion 112. In some embodiments, the second body portion 114 may be slidably engaged with the first body portion 112. In some embodiments, the second body portion 114 may be rotatably engaged with the first body portion 112. In at least some embodiments, the first body portion 112 and/or the second body portion 114 may be configured to rotate relative to each other about an axis of rotation 102.

In some embodiments, the tool 100 may comprise a release knob 160 rotatably engaged with and/or rotatably coupled to the tool body 110. In some embodiments, the release knob 160 may be configured to rotate relative to the tool body 110 and/or the first body portion 112, as discussed herein.

In some embodiments, the tool 100 may comprise a first handle portion 120 extending from the first body portion 112. In at least some embodiments, the first handle portion 120 may be fixedly attached to the first body portion 112. In some embodiments, the first handle portion 120 may be integrally formed with and/or monolithically formed with the first body portion 112.

In some embodiments, the tool 100 may comprise a second handle portion 122 extending from the second body portion 114. In at least some embodiments, the second handle portion 122 may be fixedly attached to the second body portion 114. In some embodiments, the second handle portion 122 may be integrally formed with and/or monolithically formed with the second body portion 114.

In some embodiments, the first handle portion 120 may include a first handle stop 124 extending laterally from the first handle portion 120 toward the second handle portion 122. In some embodiments, the second handle portion 122 may include a second handle stop 126 extending laterally from the second handle portion 122 toward the first handle portion 120 and/or the first handle stop 124.

In some embodiments, the first handle portion 120 may be movable relative to the second handle portion 122. In some embodiments, the first handle portion 120 may be configured to rotate about the axis of rotation 102. In some embodiments, the second handle portion 122 may be configured to rotate about the axis of rotation 102. In some embodiments, movement of the first handle portion 120 relative to the second handle portion 122 may cause and/or result in movement of the first body portion 112 relative to the second body portion 114. Contact between the first handle stop 124 and the second handle stop 126 as the first handle portion 120 is moved and/or translated toward the second handle portion 122 may prevent further movement and/or translation of the first handle portion 120 relative to the second handle portion 122. As such, the first handle stop 124 and the second handle stop 126 may act as a “hard stop” for the first handle portion 120 and the second handle portion 122.

In some embodiments, the tool 100 may comprise a first post engagement arm 130 extending from the first body portion 112. In at least some embodiments, the first post engagement arm 130 may be fixedly attached to the first body portion 112. In some embodiments, the first post engagement arm 130 may be integrally formed with and/or monolithically formed with the first body portion 112. In some embodiments, the first post engagement arm 130 may include a first arm extension 134 extending therefrom generally parallel to the axis of rotation 102. As discussed herein, the first arm extension 134 may be sized and configured to engage with the commissure post 30 of the expandable framework 12 of the replacement heart valve implant 10.

In some embodiments, the tool 100 may comprise a second post engagement arm 132 extending from the second body portion 114. In at least some embodiments, the second post engagement arm 132 may be fixedly attached to the second body portion 114. In some embodiments, the second post engagement arm 132 may be integrally formed with and/or monolithically formed with the second body portion 114. In some embodiments, the second post engagement arm 132 may include a second arm extension 136 extending therefrom generally parallel to the axis of rotation 102. The first arm extension 134 may be oriented generally parallel to the second arm extension 136. As discussed herein, the second arm extension 136 may be sized and configured to engage with the commissure post 30 of the expandable framework 12 of the replacement heart valve implant 10.

In some embodiments, the first post engagement arm 130 and/or the first arm extension 134 may be movable relative to the second post engagement arm 132 and/or the second arm extension 136. In some embodiments, the first post engagement arm 130 and/or the first arm extension 134 may be configured to rotate about the axis of rotation 102. In some embodiments, the second post engagement arm 132 and/or the second arm extension 136 may be configured to rotate about the axis of rotation 102. In some embodiments, movement of the first handle portion 120 relative to the second handle portion 122 and/or movement of the first body portion 112 relative to the second body portion 114 may cause movement of the first post engagement arm 130 and/or the first arm extension 134 relative to the second post engagement arm 132 and/or the second arm extension 136.

In some embodiments, the second body portion 114 may include a central hub 113 or a central projection extending away from the second body portion 114 along the axis of rotation 102. In at least some embodiments, the first body portion 112 may include a central opening 111 configured to receive the central hub 113 or the central projection of the second body portion 114. The central hub 113 or the central projection and the central opening 111 may cooperate to align the first body portion 112 with the second body portion 114 about the axis of rotation 102. In some embodiments, the tool 100 may include a fastener 115 configured to extend through the release knob 160 and the first body portion 112 to engage the central hub 113 or the central projection of the second body portion 114, thereby securing the release knob 160 to the tool body 110. In at least some embodiments, the fastener 115 may be a threaded fastener or a screw.

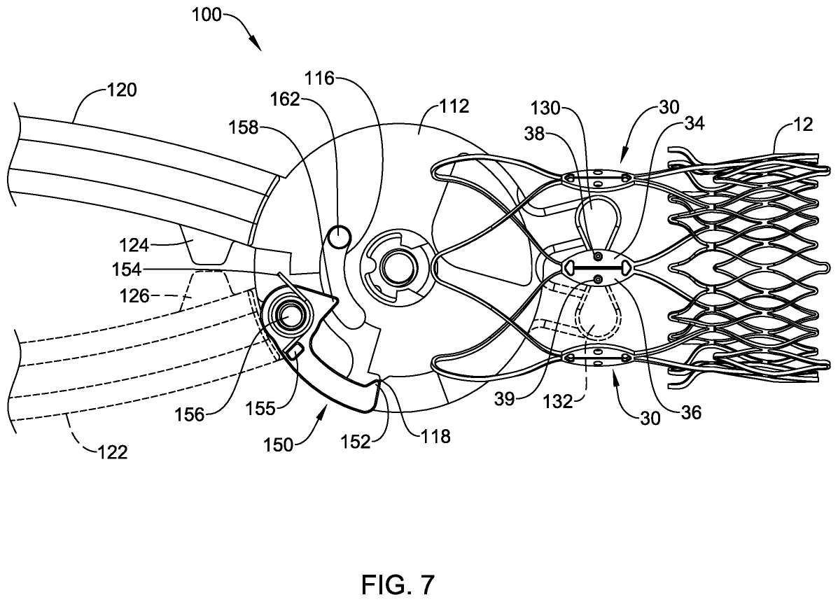

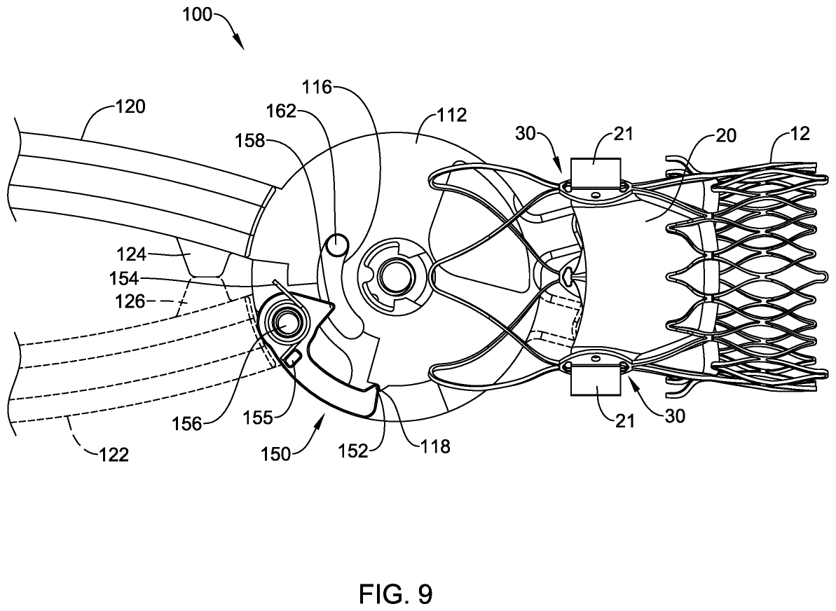

In some embodiments, the first handle portion 120 may be biased away from the second handle portion 122. In some embodiments, the tool 100 may be configured to shift between a first configuration wherein the first post engagement arm 130 and/or the first arm extension 134 and the second post engagement arm 132 and/or the second arm extension 136 are configured to engage the commissure post 30 (e.g., FIGS. 6-7) when the elongated aperture 32 is at the first circumferential dimension and a second configuration wherein the first post engagement arm 130 and/or the first arm extension 134 and the second post engagement arm 132 and/or the second arm extension 136 are configured to expand the elongated aperture 32 formed in the commissure post 30 (e.g., FIGS. 8-9) in a circumferential direction and/or toward and/or to the second circumferential dimension. In some embodiments, the tool 100 may be self-biased toward the first configuration. In some embodiments, the tool 100 may comprise a spring 140 disposed within the tool body 110. In at least some embodiments, the spring 140 may be a torsion spring. Other configurations are also contemplated. The spring 140 may be configured to engage with the first body portion 112 and the second body portion 114. The spring 140 may be configured to bias the tool 100 toward the first configuration.

In some embodiments, the tool 100 may comprise a ratchet mechanism 150 disposed within the tool body 110. In some embodiments, the ratchet mechanism 150 may be disposed between the first body portion 112 and the second body portion 114. In some embodiments, the ratchet mechanism 150 may be rotatable relative to and/or may be configured to pivot relative to the first body portion 112 and/or the second body portion 114. In some embodiments, the second body portion 114 may include a first recess configured to receive and/or engage with a ratchet spring 154. In at least some embodiments, the ratchet spring 154 may be a torsion spring. Other configurations are also contemplated. The ratchet spring 154 may be configured to engage with a ratchet projection 155 extending from the ratchet mechanism 150 generally parallel to axis of rotation 102, to bias the ratchet mechanism 150 as described herein.

In some embodiments, the first body portion 112 may include a curved slot 116 formed therein and/or extending therethrough in a direction generally parallel to the axis of rotation 102. The release knob 160 may include a release pin 162 engaged therewith and/or extending therefrom. In some embodiments, the release pin 162 may be fixedly attached to the release knob 160. In some embodiments, the release pin 162 may be releasably attached to the release knob 160, such as by friction fit, a set screw, etc. The release pin 162 may extend away from the release knob 160 toward and/or into the first body portion 112 and/or the second body portion 114. In some embodiments, the release pin 162 may extend completely through the first body portion 112 and/or the curved slot 116. In some embodiments, the release pin 162 may extend into and/or partially through the second body portion 114. Other configurations are also contemplated.

In some embodiments, the first body portion 112 may include a ratchet hub 156 extending therefrom. In some embodiments, the ratchet hub 156 may extend away from the first body portion 112 toward the second body portion 114. In some embodiments, the ratchet hub 156 may extend into the first recess and/or at least partially into the second body portion 114. The ratchet mechanism 150 may include a ratchet aperture 157 configured to engage with and/or receive the ratchet hub 156. In some embodiments, the ratchet hub 156 may be a sleeve or collar disposed on a ratchet screw configured to threadably engage the second body portion 114. In some embodiments, the ratchet hub 156 may be configured to move relative to the first body portion 112 while secured to the second body portion 114. Other configurations are also contemplated.

In some embodiments, the ratchet mechanism 150 may be configured to lock the tool 100 in the second configuration. In some embodiments, the first body portion 112 may include a body tooth 118 formed therein and/or thereon. In some embodiments, the ratchet mechanism 150 may include a ratchet tooth 152 extending therefrom. As discussed herein, the ratchet tooth 152 may be configured to releasably engage with the body tooth 118 to lock the tool 100 in the second configuration (e.g., FIG. 9).

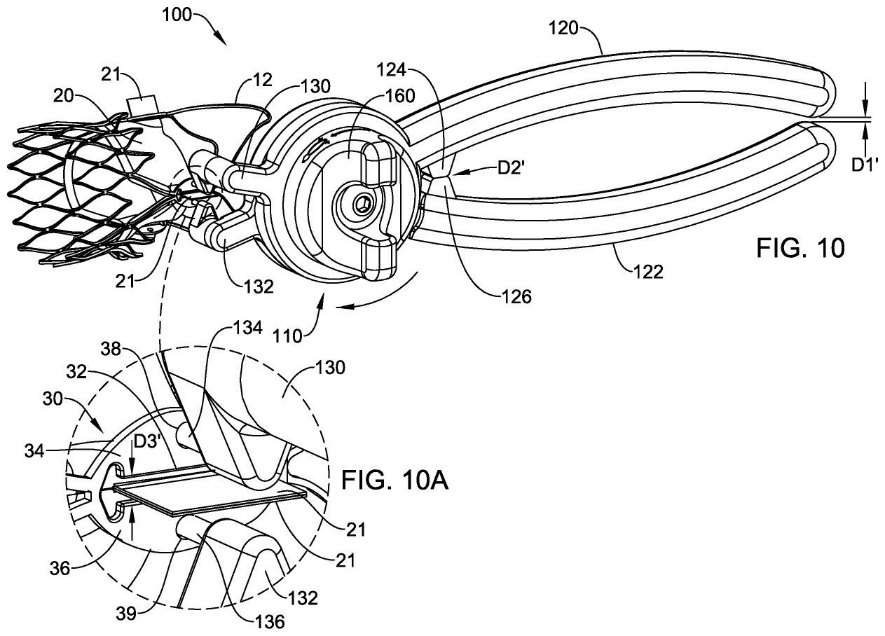

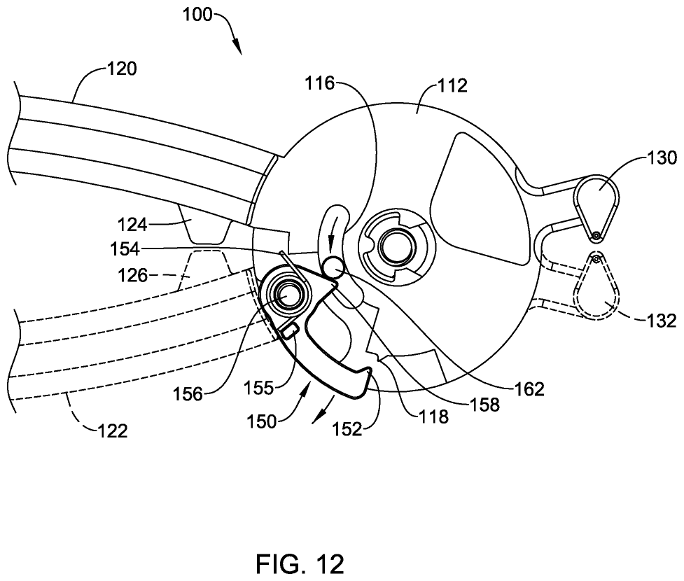

In some embodiments, the ratchet mechanism 150 may include a release projection 158 configured to engage the release pin 162. As discussed herein, rotation of the release knob 160 (e.g., FIG. 10) may cause the release pin 162 to engage the release projection 158 (e.g., FIG. 12) to disengage the ratchet mechanism 150 and/or to disengage the ratchet tooth 152 from the body tooth 118 (e.g., FIG. 12), thereby unlocking the tool 100 from the second configuration.

FIGS. 6-14 illustrate selected features, characteristics, and/or steps related to a method of assembling the replacement heart valve implant 10 using the tool 100. Some features and/or reference numbers may be shown and/or called out for reference, even if those features and/or reference numbers are not explicitly described in connection with the figure in which they appear. Some features may be described in greater detail with respect to other figures.

The method may comprise obtaining an expandable framework, such as the expandable framework 12. As discussed herein, the expandable framework 12 may include the commissure post 30 and/or a plurality of commissure posts and may define the central lumen extending axially through the expandable framework 12. The method may comprise obtaining a valve leaflet 20 including a mounting tab 21 extending therefrom. In some embodiments, the method may further comprise obtaining a second valve leaflet including a mounting tab extending therefrom and/or a plurality of valve leaflets have a mounting tab or mounting tabs extending therefrom, as described herein.

In some embodiments, the method may comprise engaging the tool 100 with the expandable framework 12 and/or the commissure post 30 of the expandable framework 12, as seen in FIGS. 6-6A. In some embodiments, the first arm extension 134 and the second arm extension 136 may be engaged with, disposed within, and/or moved into the first aperture 38 and the second aperture 39 of the commissure post 30. In some embodiments, the first arm extension 134 may be engaged with, disposed within, and/or moved into the first aperture 38 of the commissure post 30, and the second arm extension 136 may be engaged with, disposed within, and/or moved into the second aperture 39 of the commissure post 30. In some embodiments, the second arm extension 136 may be engaged with, disposed within, and/or moved into the first aperture 38 of the commissure post 30, and the first arm extension 134 may be engaged with, disposed within, and/or moved into the second aperture 39 of the commissure post 30.

In some embodiments, multiple instances of the tool 100 may be used simultaneously to engage with multiple commissure posts of the plurality of commissure posts of the expandable framework 12. For example, two tools may be engaged with two commissure posts, three tools may be engaged with three commissure posts, etc. In some alternative embodiments, a tool fixture may be provided that includes a set of first and second arm extensions for each commissure post of the expandable framework 12 in a single device. For example, the tool fixture may include three sets of first and second arm extensions configured to engage an expandable framework having three commissure posts. Other configurations are also contemplated. The tool fixture may be configured to actuate each set of first and second arm extensions individually or collectively (e.g., simultaneously).

The tool 100 may be configured to engage the commissure post 30 in the first configuration, as seen in FIGS. 6-6A. In the first configuration, a free end of the first handle portion 120 and a free end of the second handle portion 122 may be spaced apart by a first handle spacing D1. In the first configuration, the first handle stop 124 and the second handle stop 126 may be spaced apart by a first handle stop spacing D2. When engaging the tool 100 with the commissure post 30 in the first configuration, the elongated aperture 32 may define and/or may be disposed at the first circumferential dimension D3.

FIG. 7 illustrates selected aspects of the tool 100 engaged with the commissure post in the first configuration. To improve clarity, some features of the tool 100 are not shown or are shown in phantom. As seen in FIG. 7, when the tool 100 is in the first configuration, the ratchet tooth 152 of the ratchet mechanism 150 may be disposed adjacent to the body tooth 118. In some embodiments, a tip of the ratchet tooth 152 may be engaged with and/or disposed against a tip of the body tooth 118. However, in the first configuration, the ratchet mechanism 150 and/or the ratchet tooth 152 may be permitted to move relative to the body tooth 118. The ratchet spring 154 may be configured to cooperate with the ratchet projection 155 and/or the second body portion 114 (not shown) to maintain and/or urge the ratchet tooth 152 against the body tooth 118 and/or radially inward (e.g., toward the axis of rotation 102 (e.g., FIGS. 3-5).

The method may comprise translating the first post side 34 of the commissure post circumferentially away from the second post side 36 of the commissure post 30 to expand the elongated aperture 32 formed in the commissure post 30, as seen in FIGS. 8-8A, from the first circumferential dimension D3 (e.g., FIG. 6A) to the second circumferential dimension D3′ (e.g., FIG. 8A). In some embodiments, translating the first post side 34 of the commissure post 30 circumferentially away from the second post side 36 of the commissure post 30 to expand the elongated aperture 32 may include moving and/or translating the first handle portion 120 toward the second handle portion 122, as seen in FIG. 8. In some embodiments, moving and/or translating the first handle portion 120 toward the second handle portion 122 may move and/or translate the first post engagement arm 130 away from the second post engagement arm 132. In some embodiments, moving and/or translating the first handle portion 120 toward the second handle portion 122 may move and/or translate the first handle stop 124 toward the second handle stop 126. In some embodiments, moving and/or translating the first handle portion 120 toward the second handle portion 122 may move and/or translate the first handle stop 124 into contact with the second handle stop 126.

In some embodiments, translating the first post side 34 of the commissure post 30 circumferentially away from the second post side 36 of the commissure post 30 to expand the elongated aperture 32 may include shifting the tool 100 from the first configuration toward and/or to the second configuration (e.g., FIG. 8). In the second configuration, the first handle stop spacing D2 may be reduced to a second handle stop spacing D2′. In some embodiments, the second handle stop spacing D2′ may be substantially zero. In some embodiments, in the second configuration, the first handle stop 124 and the second handle stop 126 may be in contact with each other. In the second configuration, the elongated aperture 32 may be disposed at the second circumferential dimension D3′.

FIG. 9 illustrates selected aspects of the tool 100 engaged with the commissure post in the second configuration. To improve clarity, some features of the tool 100 are not shown or are shown in phantom. As seen in FIG. 9, when the tool 100 is in the second configuration, the ratchet tooth 152 of the ratchet mechanism 150 may be shifted relative to the body tooth 118 about the ratchet hub 156 and/or radially inward toward the axis of rotation 102 (e.g., FIGS. 3-5) by the ratchet spring 154. In some embodiments, the ratchet tooth 152 may be engaged with and/or disposed against a side of the body tooth 118, thereby locking the tool 100 in the second configuration (e.g., the tool 100 is prevented from automatically returning to the first configuration, regardless of bias). In the second configuration, the ratchet mechanism 150 and/or the ratchet tooth 152 may be prevented from moving relative to the body tooth 118. In the second configuration, the first handle portion 120 and the second handle portion 122 may be prevented from moving relative to each other. In the second configuration, the first body portion 112 and the second body portion 114 may be prevented from moving relative to each other. In the second configuration, the first post engagement arm 130 and the second post engagement arm 132 may be prevented from moving relative to each other. The ratchet spring 154 may be configured to cooperate with the ratchet projection 155 and/or the second body portion 114 (not shown) to maintain and/or urge the ratchet tooth 152 against the side of the body tooth 118 and/or radially inward (e.g., toward the axis of rotation 102 (e.g., FIGS. 3-5).

The method may comprise positioning the valve leaflet 20, and/or the second valve leaflet, and/or the plurality of valve leaflets within the central lumen of the expandable framework 12, as seen in FIGS. 8-9. Additionally, the method may comprise extending the mounting tab 21, and/or the second mounting tab of the second valve leaflet, and/or the mounting tabs of the plurality of valve leaflets from the central lumen radially outward through the elongated aperture 32 of the commissure post 30 (or the elongated apertures of the plurality of commissure posts) while the elongated aperture 32 is held at the second circumferential dimension D3′, as seen in FIGS. 8-9. The ratchet mechanism 150 locking the tool 100 in the second configuration makes the tool 100 easy to handle and the elongated aperture 32 remains at a fixed dimension while the mounting tab 21, and/or the second mounting tab of the second valve leaflet, and/or the mounting tabs of the plurality of valve leaflets is extended through the elongated aperture 32.

The method may comprise, after extending the mounting tab 21, and/or the second mounting tab of the second valve leaflet, and/or the mounting tabs of the plurality of valve leaflets from the central lumen radially outward through the elongated aperture 32 of the commissure post 30 (or the elongated apertures of the plurality of commissure posts) while the elongated aperture 32 is held at the second circumferential dimension D3′, unlocking the tool 100 from the second configuration. In at least some embodiments, unlocking the tool 100 from the second configuration may include, while holding the tool 100 in the second configuration (e.g., by squeezing the first handle portion 120 toward the second handle portion 122 and/or maintaining the first handle stop 124 against the second handle stop 126), actuating the release knob 160 to disengage the ratchet mechanism 150 and/or the ratchet tooth 152 from the body tooth 118, thereby unlocking the tool 100 from the second configuration, as seen in FIGS. 10 and 12. Similar to above, to improve clarity, some features of the tool 100 are not shown or are shown in phantom in FIG. 12.

In at least some embodiments, actuating the release knob 160 to disengage the ratchet mechanism 150 and/or the ratchet tooth 152 from the body tooth 118 may include rotating the release knob 160 relative to the tool body 110. In some embodiments, actuating the release knob 160 to disengage the ratchet mechanism 150 and/or the ratchet tooth 152 from the body tooth 118 may include translating the release pin 162 within the curved slot 116 and/or around the axis of rotation 102, as seen in FIG. 12. In some embodiments, translating the release pin 162 within the curved slot 116 and/or around the axis of rotation 102 may move and/or translate the release pin 162 into contact and/or engagement with the release projection 158 may overcome any bias from the ratchet spring 154 to disengage the ratchet mechanism 150 and/or to disengage the ratchet tooth 152 from the body tooth 118, thereby unlocking the tool 100 from the second configuration.

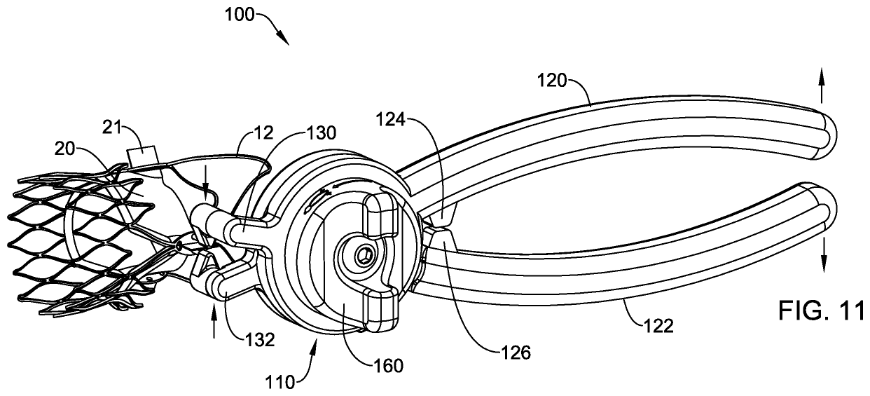

The method may comprise, after extending the mounting tab 21, and/or the second mounting tab of the second valve leaflet, and/or the mounting tabs of the plurality of valve leaflets from the central lumen radially outward through the elongated aperture 32 of the commissure post 30 (or the elongated apertures of the plurality of commissure posts) while the elongated aperture 32 is held at the second circumferential dimension D3′, and/or after unlocking the tool 100 from the second configuration, translating the first post side 34 of the commissure post 30 circumferentially toward the second post side 36 of the commissure post 30 to compress the mounting tab 21, and/or the second mounting tab of the second valve leaflet, and/or the mounting tabs of the plurality of valve leaflets between the first post side 34 and the second post side 36, as seen in FIG. 11.

In some embodiments, after unlocking the tool 100 from the second configuration, the tool 100 may be shifted toward and/or to the first configuration. In some embodiments, after unlocking the tool 100 from the second configuration, the method may comprise translating the first post side 34 of the commissure post 30 circumferentially toward the second post side 36 of the commissure post 30 to shrink the elongated aperture 32 formed in the commissure post 30 from the second circumferential dimension D3′ (e.g., FIG. 10A) toward and/or to the first circumferential dimension D3 (e.g., FIGS. 6A, 11). In some embodiments, translating the first post side 34 of the commissure post 30 circumferentially toward the second post side 36 of the commissure post 30 may include moving and/or translating the first handle portion 120 away from the second handle portion 122, as seen in FIG. 11. In some embodiments, moving and/or translating the first handle portion 120 away from the second handle portion 122 may move and/or translate the first post engagement arm 130 towards the second post engagement arm 132. In some embodiments, moving and/or translating the first handle portion 120 away from the second handle portion 122 may move and/or translate the first handle stop 124 away from the second handle stop 126 and/or the second handle stop spacing D2′ (e.g., FIG. 10) may be increased and/or shifted toward and/or to the first handle stop spacing D2 (e.g., FIGS. 6A, 11).

In some embodiments, after translating the first post side 34 of the commissure post 30 circumferentially towards the second post side 36 of the commissure post 30 and/or shifting the tool 100 from the second configuration toward and/or to the first configuration, the mounting tab 21, and/or the second mounting tab of the second valve leaflet, and/or the mounting tabs of the plurality of valve leaflets may be secured to the commissure post 30 and/or the plurality of commissure posts only by compression. Due to the relative sizing and/or dimensions of the interacting features and/or elements of the replacement heart valve implant 10, no additional securement and/or attachment means may be necessary to secure the valve leaflet 20, the second valve leaflet, and/or the plurality of valve leaflets and/or the mounting tab 21, the second mounting tab, and/or the mounting tabs thereof to the expandable framework 12, the commissure post 30, and/or the plurality of commissure posts.

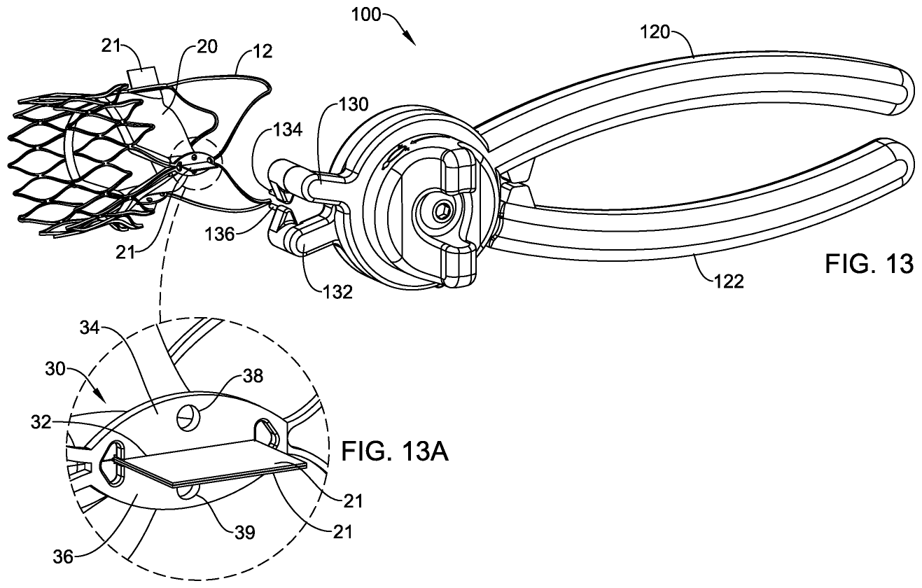

In some embodiments, after translating the first post side 34 of the commissure post circumferentially towards the second post side 36 of the commissure post 30 and/or shifting the tool 100 from the second configuration toward and/or to the first configuration, the method may comprise disengaging the tool 100 from the expandable framework 12 and/or the commissure post 30, as seen in FIGS. 13-13A. In some embodiments, after translating the first post side 34 of the commissure post 30 circumferentially towards the second post side 36 of the commissure post 30 and/or shifting the tool 100 from the second configuration toward and/or to the first configuration, the method may comprise disengaging the first arm extension 134 and the second arm extension 136 from the expandable framework 12, the commissure post 30, and/or the first aperture 38 and the second aperture 39.

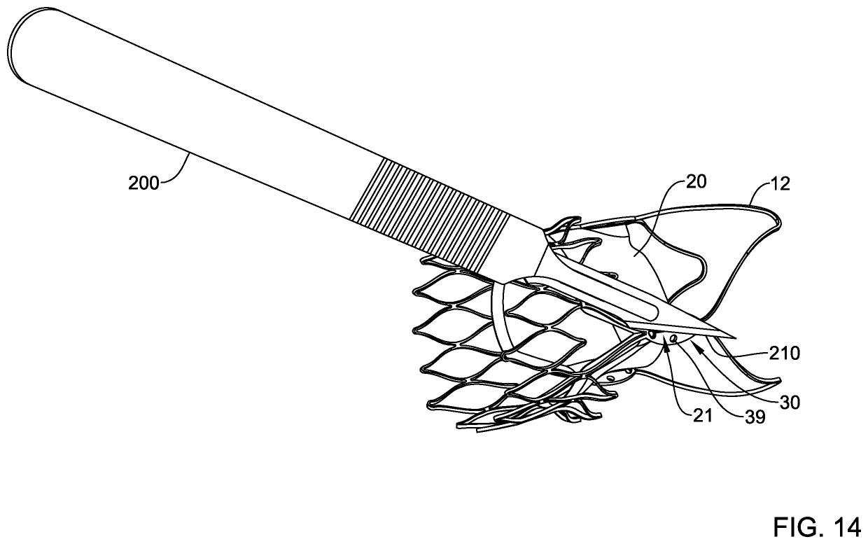

In at least some embodiments, after disengaging the tool 100 from the expandable framework 12 and/or the commissure post 30, the method may comprise trimming a portion of the mounting tab 21, the second mounting tab, and/or the mounting tabs extending radially outward of the commissure post 30 and/or the plurality of commissure posts using a second tool 200, as seen in FIG. 14. In some embodiments, the second tool 200 may be a scalpel or another cutting tool having a cutting edge 210 adapted and configured to cut the mounting tab 21, the second mounting tab, and/or the mounting tabs without tearing the mounting tab 21, the second mounting tab, and/or the mounting tabs.

In some embodiments, the mounting tab 21, the second mounting tab, and/or the mounting tabs may be trimmed at a distance of three thicknesses of the mounting tab 21, the second mounting tab, and/or the mounting tabs or less from the commissure post 30 and/or the plurality of commissure posts. In some embodiments, the mounting tab 21, the second mounting tab, and/or the mounting tabs may be trimmed at a distance of two thicknesses of the mounting tab 21, the second mounting tab, and/or the mounting tabs or less from the commissure post 30 and/or the plurality of commissure posts. In some embodiments, the mounting tab 21, the second mounting tab, and/or the mounting tabs may be trimmed at a distance of one thickness of the mounting tab 21, the second mounting tab, and/or the mounting tabs or less from the commissure post 30 and/or the plurality of commissure posts. Other configurations are also contemplated. It should be noted that in FIG. 1, the mounting tab 21, the second mounting tab, and/or the mounting tabs are shown with extra length extending radially outward of the commissure post 30 and/or the plurality of commissure posts. The features of the disclosure may permit the mounting tab 21, the second mounting tab, and/or the mounting tabs to be trimmed much closer to the commissure post 30 and/or the plurality of commissure posts than shown in FIG. 1. Note that in FIG. 14, the mounting tab 21, the second mounting tab, and/or the mounting tabs, after trimming, does not overlap the second aperture 39.

It will be understood that the disclosed method and/or method steps may not result in the replacement heart valve implant 10 being complete and/or ready for implantation. In some cases, the disclosed method and/or method steps may be a part or a portion of an overall assembly process. Some additional steps may be required to assemble additional elements and/or components before completing the replacement heart valve implant 10. In the interest of brevity, those additional steps may be considered to be known and are therefore omitted from the disclosure.

The benefits afforded by the current disclosure may be numerous. For example, the replacement heart valve implant 10 may be devoid of adhesives, sutures, and/or other fastening elements typically used to secure the valve leaflet(s) and/or the mounting tab(s) to the expandable framework, thereby reducing overall cost and time to manufacture or assembly the replacement heart valve implant 10. In another example, eliminating sutures or other fastening element may reduce the number of holes in and/or piercings made in the valve leaflet(s) and/or the mounting tab(s), thereby reducing the occurrence of and/or the possible occurrence of cracks, tears, stress fractures, or other failings. In another example, the replacement heart valve implant 10 may have less tissue disposed on and/or around the commissure post(s), which may positively affect compression, loading, and/or recapture of the replacement heart valve implant 10. In another example, less tissue disposed on and/or around the commissure post(s) may permit a smaller delivery device to be used. Other benefits are also contemplated and will be apparent.

The materials that can be used for the various components of the replacement heart valve implant, the various elements thereof, and/or the tool disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the valve leaflet(s), the mounting tab(s), the tool, the tool body, the first and second handle portions, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, sulfasalazine, estrogen, and mesalamine);

antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve implant, comprising:
an expandable framework including a commissure post and defining a central lumen extending axially through the expandable framework; and
a valve leaflet disposed within the central lumen of the expandable framework, the valve leaflet including a mounting tab extending therefrom;
wherein the mounting tab is secured to the expandable framework at the commissure post;
wherein the commissure post is configured to hold the mounting tab in compression.

2. The replacement heart valve implant of claim 1, wherein the expandable framework is formed from a resilient shape memory material.

3. The replacement heart valve implant of claim 1, wherein the commissure post includes an elongated aperture at least partially defined by a first post side and a second post side, the elongated aperture having an axial extent and a circumferential extent, the axial extent being greater than the circumferential extent.

4. The replacement heart valve implant of claim 3, wherein the mounting tab extends through the elongated aperture.

5. The replacement heart valve implant of claim 3, wherein the circumferential extent is configured to be shifted between a first circumferential dimension between the first post side and the second post side, and a second circumferential dimension between the first post side and the second post side, wherein the second circumferential dimension is greater than the first circumferential dimension.

6. The replacement heart valve implant of claim 5, wherein the commissure post is self-biased toward the first circumferential dimension.

7. The replacement heart valve implant of claim 5, wherein the second circumferential dimension is greater than a thickness of the mounting tab.

8. The replacement heart valve implant of claim 1, further comprising a second valve leaflet disposed within the central lumen of the expandable framework, wherein the second valve leaflet includes a second mounting tab extending therefrom.

9. The replacement heart valve implant of claim 8, wherein the second mounting tab is secured to the expandable framework at the commissure post;
wherein the commissure post is configured to hold the second mounting tab in compression.

10. The replacement heart valve implant of claim 9, wherein the mounting tab and the second mounting tab are in intimate contact with each other at the commissure post.

11. A tool for assembling a replacement heart valve implant, comprising:
a tool body comprising a first body portion and a second body portion rotatably engaged with the first body portion;
a first handle portion extending from the first body portion, the first handle portion being configured to rotate about an axis of rotation;
a second handle portion extending from the second body portion, the second handle portion being configured to rotate about the axis of rotation;
a first post engagement arm extending from the first body portion, the first post engagement arm being configured to rotate about the axis of rotation; and
a second post engagement arm extending from the second body portion, the second post engagement arm being configured to rotate about the axis of rotation;
wherein translation of the first handle portion toward the second handle portion translates the first post engagement arm away from the second post engagement arm.

12. The tool of claim 11, wherein the first handle portion is biased away from the second handle portion.

13. The tool of claim 11, wherein the tool is configured to shift between a first configuration wherein the first post engagement arm and the second post engagement arm are configured to engage a commissure post of an expandable framework of the replacement heart valve implant, and a second configuration wherein the first post engagement arm and the second post engagement arm are configured to expand an elongate aperture formed in the commissure post in a circumferential direction.

14. The tool of claim 13, wherein the tool is self-biased toward the first configuration.

15. The tool of claim 14, further comprising a spring disposed within the tool body, wherein the spring biases the tool toward the first configuration.

16. The tool of claim 14, further comprising a ratchet mechanism configured to lock the tool in the second configuration.

17. The tool of claim 16, further comprising a release knob rotatably engaged with the tool body, the release knob being configured to disengage the ratchet mechanism, thereby unlocking the tool from the second configuration.

18. A method of assembling a replacement heart valve implant, comprising:
obtaining an expandable framework including a commissure post and defining a central lumen extending axially through the expandable framework;
obtaining a valve leaflet including a mounting tab extending therefrom;
translating a first post side of the commissure post circumferentially away from a second post side of the commissure post to expand an elongated aperture formed in the commissure post from a first circumferential dimension to a second circumferential dimension;
positioning the valve leaflet within the central lumen;
extending the mounting tab from the central lumen radially outward through the elongated aperture while the elongated aperture is held at the second circumferential dimension; and
translating the first post side of the commissure post circumferentially toward the second post side of the commissure post to compress the mounting tab between the first post side and the second post side.

19. The method of claim 18, wherein after translating the first post side of the commissure post circumferentially toward the second post side of the commissure post, the mounting tab is secured to the commissure post only by compression.

20. The method of claim 18, further comprising trimming a portion of the mounting tab extending radially outward of the commissure post.
